(19) United States
(12) Patent Application Publication
(10) Pub. No.: US 2019/0148002 A1
(43) Pub. Date: May 16, 2019

(54) SYSTEM FOR AFFECTING BEHAVIOR OF A SUBJECT

(71) Applicants: Nancy NUNES, Plainville, MA (US); J. Christopher FLAHERTY, Auburndale, FL (US)

(72) Inventors: Nancy NUNES, Plainville, MA (US); J. Christopher FLAHERTY, Auburndale, FL (US)

(21) Appl. No.: 16/093,314

(22) PCT Filed: Apr. 14, 2017
(86) PCT No.: PCT/US2017/027605

§ 371 Date: Oct. 12, 2018

Related U.S. Application Data
(60) Provisional application No. 62/323,646, filed on Apr. 16, 2016.

(51) Int. Cl. G16H 20/70; G06F 3/01

(52) U.S. Cl. CPC G16H 20/70; G06F 3/011

(57) ABSTRACT

A system for modifying behavior of a subject is provided. The system comprises: an input assembly configured to receive input data; a data analyzer configured to receive the input data from the input assembly, analyze the input data and produce a behavioral modification regimen based on the input data; and an output assembly configured to receive the behavioral modification regimen from the data analyzer and produce output data based on the behavioral modification regimen. The output data is configured to modify behavior of the subject. Methods of modifying behavior of a subject are also provided.

### RELATED APPLICATIONS

This application claims the benefit of U.S. Provisional Application Ser. No. 62/323,646, titled “System for Affecting Behavior of a Subject”, filed Apr. 16, 2016, the content of which is incorporated herein by reference in its entirety for all purposes.

### TECHNICAL FIELD

### Field

Inventive concepts relate generally to behavioral modification systems and methods, and more particularly to systems and methods which provide or modify media being viewed or otherwise experienced to alter behavior.

### BACKGROUND

People may behave in ways which are undesirable as a result of their conditioning, such as conditioning that is influenced by exposure to media. For example, individual experience can affect a person's confidence, a person's ability to provide fair service, or a person's ability to recognize, react to or avoid inappropriate treatment of themselves or others. There is a need for systems that can positively modify or maintain a person's behavior.

### SUMMARY

According to an aspect, a system for modifying behavior of a subject is provided. The system comprises: an input assembly configured to receive input data a data analyzer configured to receive the input data from the input assembly, analyze the input data and produce a behavioral modification regimen based on the input data; and an output assembly configured to receive the behavioral modification regimen from the data analyzer and produce output data based on the behavioral modification regimen. The output data is configured to modify behavior and/or maintain behavior of the subject.

In some embodiments, the system comprises a first communication device at a location of the subject, and a second communication device at a second location. The first communication device can comprise a device selected from the group consisting of: computer; laptop computer; tablet computer, phone; smartphone; television; smart television; radio; music playing device; watch; virtual reality glasses; virtual reality goggles, a 3D camera; a holographic virtual reality device; an enterable theatre device; a video player; an audio player, a wearable device; a device proximate the subject while sitting; an implantable device; and combinations thereof. The first communication device can comprise the input assembly. The first communication device can comprise the output assembly. The first communication device can comprise the data analyzer. The second communication device can comprise a device selected from the group consisting of: computer, network; CPU; and combinations thereof. The second communication device can comprise the input assembly. The second communication device can comprise the data analyzer. The first communication device and the second communication device can communicate via a network. The network can comprise a network selected from the group consisting of: internet; wide area network; local area network; cellular service network; wired network; wireless network; and combinations thereof. The first communication device and the second communication device can communicate via a connection selected from the group consisting of: wired connection; wireless connection; Bluetooth connection; sneakernet; push and/or pull data transfer; and combinations thereof.

In some embodiments, the system comprises a first communication device at a location of the subject. The first communication device can comprise a device selected from the group consisting of: computer, laptop computer, tablet computer, phone; smartphone; television; smart television; radio; music playing device; watch; virtual reality glasses; virtual reality goggles; a 3D camera; a holographic virtual reality device; an enterable theatre device; a video player; an audio player; a wearable device; a device proximate the subject while sitting; an implantable device; and combinations thereof. The first communication device can comprise one or more of: the input assembly; the data analyzer; and the output assembly. The first communication device can comprise two or more of: the input assembly; the data analyzer; and the output assembly. The first communication device can comprise the data analyzer. The data analyzer can comprise a software program loaded onto the first communication device. The system can further comprise a second communication device in communication with the first communication device, and the second communication device can be configured to transfer data to and/or from the first communication device. The second communication device can be at a location remote from the subject. The first communication device can comprise the input assembly and the output assembly, and the second communication device can comprise the data analyzer. The second communication device can comprise a device selected from the group consisting of: computer, network; internet; cloud; disk drive; electronic file storage device; electronic library; a living tissue information storage device; and combinations thereof.

In some embodiments, the system is configured to operate in a closed-loop fashion.

In some embodiments, the subject comprises a single person.

In some embodiments, the subject comprises a group of people.

In some embodiments, the subject comprise one or more people selected from the group consisting of: adolescent; child; adult; clinical patient; person in the customer service industry; person in the law enforcement industry; inmate; self-help consumer; educator; service provider; healthcare provider, medical provider, juror; judge; legal counsel; an entity that is an enemy and/or adversary of another entity; a radicalized person or group; a harasser of others; a bully such as an anonymous bully; and combinations thereof. In some embodiments, the subject comprises two or more people selected from the group consisting of: adolescent; child; adult; clinical patient; person in the customer service industry; person in the law enforcement industry; inmate; self-help consumer; educator; service provider, healthcare provider; medical provider; juror, judge; legal counsel; an entity that is an enemy and/or adversary of another entity; a radicalized person or group; a harasser of others; a bully such as an anonymous bully; and combinations thereof.

In some embodiments, the user input assembly comprises an assembly selected from the group consisting of: microphone; mouse; keyboard; touchscreen; camera; eye tracking device; joystick; trackpad; sip and puff device; gesture tracking device; brain machine interface; any computer input device; and combinations thereof.

In some embodiments, the input assembly comprises an active component.

In some embodiments, the input assembly comprises a passive component.

In some embodiments, the input assembly is configured to receive the input data from a user. The user can comprise at least the subject. The user can further comprise a second user. The user can comprise a parent of the subject. The user can comprise a clinician of the subject such as a psychiatrist or psychologist treating the subject. The user can comprise the employer of the subject. The user can comprise a behavioral modification group.

In some embodiments, the input data comprises data recorded by the input assembly. The input data can comprise data recorded from the environment of the subject. The input data can comprise data selected from the group consisting of: text read by subject; verbal communications heard by subject; music heard by subject; media viewed by subject; purchase patterns of the subject (e.g. internet purchase patterns); holographically presented data; simulated data; a manipulated sensory experience; and combinations thereof.

In some embodiments, the input data comprises bias data of the subject. The bias data can comprise prejudice bias data of the subject. The bias data can comprise bias data related to a parameter selected from the group consisting of: age; race; ethnicity; nationality; religion; gender, gender identity; height; weight; sexual preference; posture; a mobility pattern; a disability; a physical characteristic; an appearance characteristic; hair style or other grooming characteristic; apparel; genetic makeup; phenotype; medical history; speech pattern; vocal pattern; a gesture; a possession of the subject such as phone, car, smart device, clothing, and/or jewelry; and combinations thereof. The bias data can comprise data extracted from a work and/or living environment of the subject. The bias data can comprise data extracted from media viewed by the subject. The bias data can comprise data extracted from purchase patterns of the subject (e.g. internet purchase patterns). The media can comprise media selected from the group consisting of: television; radio broadcast; web site; podcast; music; advertisement; digital media; analog media; sensory media; media presented in an application; media comprising gaming and/or other entertainment content; purchased media; subscription media; broadcast media; and combinations thereof. The data analyzer can be configured to quantify the bias data. The output data can be configured to reduce the effect of the bias data on the subject.

In some embodiments, the data analyzer is configured to identify self-limiting behavior.

In some embodiments, the data analyzer is configured to determine the duration of exposure of the output data to the subject.

In some embodiments, the data analyzer is configured to determine the frequency of exposure of the output data to the subject.

In some embodiments, the data analyzer is configured to assess effectiveness of the behavior modification on the subject. The data analyzer can be configured to use artificial intelligence and/or computer learning to improve efficacy of the behavior modification over time.

In some embodiments, the data analyzer comprises an algorithm configured to provide security to the system. The algorithm can assess the input data, historical data and/or the output data for integrity. The algorithm can compare the assessed data to a data set of acceptable values.

In some embodiments, the data analyzer comprise an algorithm configured to differentiate data received from a first subject from data received from a second subject. The algorithm can be configured to stratify the received data, such as to separately analyze one or more subjects from a group of subjects from another one or more subjects from the group of subjects. The algorithm can be configured to recommend separation of one or more subjects from the group. The algorithm can be configured to analyze data from a subset of subjects of the group. The system can be configured to provide improved behavioral modification to the one or more separated subjects of the group and/or to provide different output data to the one or more separated subjects of the group.

In some embodiments, the data analyzer comprises an algorithm configured to assess a first set of data and produce a first set of results, and to assess a second set of data and produce a second set of results. The algorithm can compare, combine and/or otherwise further process the first result set and the second result set.

In some embodiments, the output data comprises a behavioral modification regimen configured to modify prejudice and/or implicit bias of the subject.

In some embodiments, the output data comprises a behavioral modification regimen configured to reduce, maintain and/or prevent creation of a detrimental bias of the subject.

In some embodiments, the output data comprises a behavioral modification regimen configured to modify self-limiting behavior of the subject.

In some embodiments, the output data comprises a behavioral modification regimen configured to identify harmful interactions of the subject with other people.

In some embodiments, the output data behavioral modification regimen is configured to identify harmful interactions of subject with an environment.

In some embodiments, the output data comprises a behavioral modification regimen configured to improve confidence of the subject.

In some embodiments, the output assembly includes at least one output component selected from the group consisting of: display; touchscreen; audio transducer; speaker; vibrational transducer; light; and combinations thereof.

In some embodiments, the output assembly comprises a display, and wherein the output data comprises visible output data presented on the display.

In some embodiments, the output assembly comprises an audio transducer, and wherein the output data comprises audio output data presented by the audio transducer.

In some embodiments, the output data comprises data selected from the group consisting of: information; game; media; report of current event; historical article; online interaction; interaction with a behavioral modification caregiver; holographically presented data; simulated data; a manipulated sensory experience; and combinations thereof.

In some embodiments, the output data comprises a recommendation of an external behavioral modification service.

In some embodiments, the output data comprises behavioral modification progress reports.

In some embodiments, the output data is configured to inform the subject of self-limiting behavior.

In some embodiments, the output data comprises a report. The report can comprise: an assessment of behavioral modification of a subject; an assessment of actual use of one or more behavioral modification regimens; and/or an assessment of a bias quotient.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a schematic view of a system for modifying behavior of a subject, including a communication device and a data analyzer, consistent with the present inventive concepts.

FIG. 2 illustrates a schematic view of a system for modifying behavior of a subject, including multiple communications devices attached to a media access device, consistent with the present inventive concepts.

FIG. 3 illustrates a flow chart of a method for producing output data configured to modify behavior of a subject based on an analysis of input data, consistent with the present inventive concepts.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The terms “connection”, “connected”, “connecting” and the like, where used herein, are to be taken to include any type of connection between two or more components. The connection can include an operable connection which allows multiple connected components to operate together such as to transfer information, power and/or material between the components. An operable connection can include a physical connection, such as a physical connection including one or more wires, optical fibers, wave guides, or other electrical, optical or mechanical connection. Alternatively or additionally, an operable connection can include a non-physical or “wireless” connection, such as a wireless connection in which information and/or power is transmitted between components using electromagnetic energy. A connection can include a connection selected from the group consisting of: a wired connection; a wireless connection; an electrical connection; a mechanical connection; an optical connection; a sound propagating connection; a fluid connection; and combinations of one or more of these.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

If a person is aware of a negative predisposition, they can consciously choose to behave in a desirable manner, especially when they are afforded sufficient time to recognize the potential influence of their conditioning. The present inventive concepts include systems that are configured to modify and/or maintain (“modify” herein) the behavior of a subject based on data input into a device such as a computer or other data input device. These systems can be configured to raise awareness of harmful conditioning, and can be configured to recondition a subject for desired behavior, even in stressful, reactionary situations. In some embodiments, the system is configured to reduce a negative bias or other otherwise modify behavior of the subject in a desired manner. In some embodiments, the modification of behavior includes maintaining existing behavior that is at an acceptable level (e.g. preventing degradation to an unacceptable level). The subject can include a single person or a group of people. In some embodiments, the subject comprises one or more people selected from the group consisting of: adolescent; child; adult; clinical patient; person in the customer service industry; person in the law enforcement industry; inmate; self-help consumer; educator; service provider, healthcare provider, medical provider; juror, judge; legal counsel; an entity that is an enemy and/or adversary of another entity; a radicalized person or group; a harasser of others; a bully such as an anonymous bully; and combinations of one, two or more of these.

As used herein, the term “bias” shall include an association between a demographic attribute and a trait of the subject. Bias can be measured and quantified (e.g. to indicate a level from slight to strong). A trait can be classified with a positive, negative or neutral rating, as may be desirable to a subject (or to all subjects). Traits can include “anti-traits”, where exposure to an anti-trait can reduce a bias rating of the trait. In some embodiments, a rating can include a confidence or integrity level associated with the rating. In some embodiments, a rating is formed and/or assessed via crowd sourcing. In some embodiments, a system of the present inventive concepts includes a security algorithm configured to prevent tampering or other undesired modification to the system (intentional or unintentional).

The system of the present inventive concepts can be configured to detect and quantify behavior, and can be configured to detect and quantify levels of bias of a subject. The system can be configured to provide output (e.g. exposures to media or live events) which can modify conditioned bias and behavior of a subject to a desired level.

In instances in which a subject has a negative bias against the demographic to which they belong, the subject may exhibit self-limiting behavior. The system of the present inventive concepts can present media depicting a role model from the target demographic. For example, the media can portray the role model behaving in a manner which demonstrates the desired bias. In addition, output of the system can include suggested activities or other specific experiences configured to expose the subject to experiences which include the desired bias (e.g. where the subject participates in or observes the desired bias exhibiting behavior in a live forum). For example, the subject can include an elderly person who shows a strong association between old and unhealthy, and the system can be configured to weaken that association by providing media showcasing healthy active senior citizens, by providing an interaction with a person who is healthy and active, and/or by providing or prescribing an experience to highlight the health of the subject (e.g via social networking).

In instances in which the subject is exposed to a news story reporting an individual of a particular demographic (e.g. appearance, race or age) committing a crime, the subject may develop an association between that demographic and crime. The system of the present inventive concepts can be configured to present media depicting that demographic in a positive role, such as a care giving role, to balance the negative exposure to the demographic and thus neutralize or diminish the association of the negative trait with that particular demographic.

The system of the present inventive concepts can be configured to measure bias levels before and after exposure to media or other experiences, such as to create correlations between those experiences and the measured effect on bias (e.g. for future use with this and/or other subjects). The system can be configured to adjust and/or recommend different types of exposure to media or other experiences for subjects, and subsequently measure levels after those system-driven exposures to similarly analyze changes to the subject's bias (e.g. in order to improve the effectiveness of the system in a closed-loop fashion).

The system of the present inventive concepts can be configured to measure one or more particular set of biases for a subject, and present those measurements to the subject, such as to allow the subject to consciously decide to behave in a different manner (e.g. to behave in a more desirable or other different manner after being made aware of the bias measurements).

The system of the present inventive concepts can be configured to rate content for bias generation, such as a rating based on measured bias and behavior. Alternatively or additionally, the system can be configured to receive a content rating for bias content, such as by receiving a rating based on crowd sourced ratings. The system can be configured to use “as is” content which has a rating above a threshold. The system can be configured to alter content to manipulate a bias rating, such as for presentation to a group. For example, the system can be configured to replace the pronoun “he” or “she” with the gender-neutral “ze”, such as to create a gender neutral association.

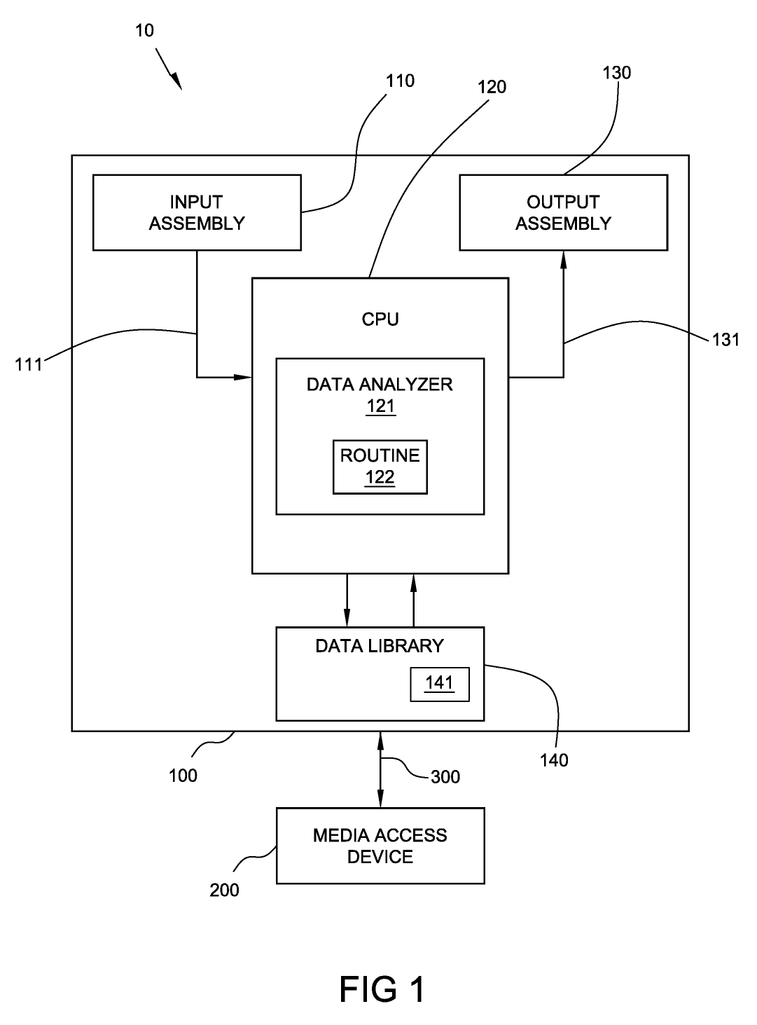

Referring now to FIG. 1, a system for modifying behavior of a subject and including multiple communications devices attached to a media access device is illustrated, consistent with the present inventive concepts. System 10 comprises one or more communication devices, such as communication device 100 shown. In some embodiments, system 10 can comprise a media access device, such as media access device 200 which can transfer data to and/or from communication device 100 via data communication link 300.

Communication device 100 can comprise one, two or more devices selected from the group consisting of: computer; laptop computer; tablet computer; phone; smartphone; television; smart television; radio; music playing device; watch; virtual reality glasses; virtual reality goggles; a 3D camera; a holographic virtual reality device; an enterable theatre device; a video player, an audio player, a wearable device; a device proximate the subject while sitting; an implantable device; and combinations of one, two or more of these. Communication device 100 can comprise an input assembly 110, which can comprise one or more assemblies selected from the group consisting of: microphone; mouse; keyboard; touchscreen; camera; eye tracking device; joystick; trackpad; sip and puff device; gesture tracking device; brain machine interface; any computer input device; and combinations of one, two or more of these. In some embodiments, input assembly 110 comprises an active component, such as a keyboard or mouse controlled by a subject (e.g. a single person or a group of people). Alternatively or additionally, input assembly 110 can comprise a passive component, such as a microphone, camera, GPS tracking element; remote data storage device; or other device which does not necessarily require action by the subject. Input data 111 recorded by input assembly 110 can comprise data selected from the group consisting of: text read by subject; verbal communications heard by subject; music heard by subject; media viewed by subject; purchase patterns of the subject (e.g. internet purchase patterns); holographically presented data; simulated data; a manipulated sensory experience; and combinations thereof. Input data 111 can comprise data received from any user of system 10, such as a user comprising a person or people selected from the group consisting of: a parent of the subject; a clinician of the subject such as a psychiatrist or psychologist treating the subject; an employer of the subject; a behavioral modification group; and combinations of one, two or more of these.

Communication device 100 can further comprise CPU 120 which receives input data 111 from input device 110. CPU 120 comprises one or more components or algorithms to analyze input data 111, such as data analyzer 121 and routine 122. Communication device 100 can further comprise output assembly 130 which receives data from CPU 120 and presents the data (e.g. output data 131) to the subject. Output assembly 130 can comprise one or more output components selected from the group consisting of: display; touchscreen; audio transducer, speaker, vibrational transducer, light; and combinations of one, two or more of these. Output assembly 130 can comprise a display, and output data 131 can comprise visible output data presented to the subject on the display. Output assembly 130 can comprise an audio transducer (e.g. a speaker), and output data 131 can comprise audio output data presented to the subject by the audio transducer. Output data 131 can comprise data selected from the group consisting of: game; media; report of current event; historical article; online interaction; interaction with a behavioral modification caregiver; holographically presented data; simulated data; a manipulated sensory experience; and combinations thereof. Output data 131 can comprise one or more of: a recommendation of an external behavioral modification service; behavioral modification progress reports; feedback to the subject regarding self-limiting behavior; and combinations of one, two or more of these. Output data 131 can include a report, such as a report that provides an assessment of behavioral modification of a subject and/or a report that provides actual use of one or more behavioral modification regimens.

Data analyzer 121 can be configured to analyze input data 111 received from input assembly 110, such as data analyzed by one or more algorithms of routine 122. Routine 122 can comprise one or more software programs, such as one or more software programs installed into (e.g. by a subject or other user of system 10) data analyzer 121 of communication device 100. In some embodiments, CPU 120 can comprise a data library 140 which can include historical data 141. Historical data 141 can comprise data previously received from an input assembly 110 and/or one or more pre-determined data sets (e.g. historical data collected other subjects and/or other data). In these embodiments, data analyzer 121 can be further configured to analyze data stored in data library 140.

In some embodiments, routine 122 comprises an algorithm configured to provide security to system 10, as described hereabove. In some embodiments, the algorithm assesses input data 111, historical data 141 and/or output data 131 for integrity, such as to compare to a pre-determined data set of acceptable values (e.g. pre-determined data stored in data library 140).

In some embodiments, routine 122 comprises an algorithm configured to differentiate data (e.g. input data 111 and/or historical data 141) received from a first subject from data received from a second subject (e.g. when the subject comprises multiple people). In these embodiments, routine 122 can be configured to stratify the data, such as to analyze (e.g. separately analyze) one or more subjects from a group of subjects from another one or more subjects from the group. In these embodiments, routine 122 can be configured to recommend separation of one or more subjects from the group (e.g. to analyze data from a subset of a group), such as to provide improved behavioral modification to one or more subsets of subjects (e.g. the separated subjects), and/or to create different output data 131 for one or more subsets of subjects (e.g. different than what is provided to the entire group or different than what is provided to other subjects of the group). System 10 can require confirmation (e.g. confirmation input into input assembly 110) from one or more users of system 10 prior to separating the one or more subjects from the group.

In some embodiments, routine 122 comprises an algorithm configured to assess a first set of data (e.g. input data 111 and/or historical data 141) and produce a first set of results, and then to assess a second set of data and produce a second set of results. In these embodiments, the first result set and the second result set can be compared, combined, and/or otherwise further processed.

Analysis performed by data analyzer 121 can include an analysis selected from the group consisting of: mathematically processing the data (e.g. input data 111 and/or historical data 141); qualitatively assessing the data; comparing the data to a threshold; comparing input data 111 to historical data 141; removing data from historical data 141 (e.g. removing data based on a security check and/or removing data based on removing a subject from a group of subjects); and combinations thereof.

Analysis performed by data analyzer 121 can include an analysis selected from the group consisting of: identifying key words in data (e.g. input data 111 and/or historical data 141); identifying a particular tone in input data 111 comprising recorded speech (e.g. tone of the subject or a person in the subject's environment); identifying a particular tone in media (e.g. a recorded song or media broadcast) heard by the subject; identifying a parameter of a phrase; identifying a parameter of a phrase pattern; identifying an accent or dialect (e.g. associated with a demographic); identifying a repeated characteristic in data (e.g. a repeated characteristic of a single subject that is creating an undesired bias in data analysis); and combinations thereof.

In some embodiments, data analyzer 121 is configured to receive input data 111 from an input assembly 110 comprising a microphone (e.g. a microphone of a computer or a smart device such as a cell phone or tablet), such as when input assembly 110 records speech of the subject (e.g. when data analyzer 121 converts speech to text or analyzes tone and/or volume of the speech) or when input assembly 110 records sounds present in the environment of the subject (e.g. music or other media heard by the subject). Alternatively or additionally, input assembly 110 can comprise a camera configured to record images from the subject's environment, such as when data analyzer 121 analyzes the images. In some embodiments, input assembly 110 comprises the subjects phone (e.g. cell phone).

In some embodiments, input assembly 110 records and data analyzer 121 analyzes data selected from the group consisting of: text read by subject; verbal communications heard by subject; music heard by subject; media viewed by subject; media attended by subject (e.g. a play, a sporting event, a rally, a protest); purchase patterns of the subject (e.g. internet purchase patterns); holographically presented data; simulated data; a manipulated sensory experience; and combinations thereof. In some embodiments, input data 111 comprises bias data of the subject, such as prejudice bias data of the subject. Input data 111 (e.g. as recorded by input assembly 110) can comprise bias data related to a parameter selected from the group consisting of: age; race; ethnicity; nationality; religion; gender; gender identity; height; weight; sexual preference; posture; a mobility pattern; a disability; a physical characteristic; an appearance characteristic; hair style or other grooming characteristic; apparel; genetic makeup; phenotype; medical history; speech pattern; vocal pattern; a gesture; a possession of the subject such as phone, car, smart device, clothing, and/or jewelry; and combinations thereof. Input assembly 110 can be configured to record data from a work environment and/or living environment of the subject and/or data extracted from media viewed by the subject (e.g. media such as television; radio broadcast; web site; podcast; music; advertisement; digital media; analog media; sensory media; media presented in an application; media comprising gaming and/or other entertainment content; purchased media; subscription media; broadcast media; and combinations thereof). Input assembly 110 can be configured to record data related to purchase patterns of the subject, such as data related to purchases made via the internet. In some embodiments, data analyzer 121 quantifies bias data. In some embodiments, output data 131 is configured to reduce the effect of the bias data on the subject.

In some embodiments, data analyzer 121 is configured to analyze a parameter selected from the group consisting of: body language; facial expression; breathing pattern; pupil dilation; heart rate; body temperature; electromagnetic energy level; mood; a measurement of health; and combinations thereof. In some embodiments, data analyzer 121 is configured to analyze data recorded by one or more of: a video or other camera; data self-reported by the subject; data reported by a family member of the subject; data reported by a health-care provider of the subject (e.g. a doctor, nurse or therapist); and combinations of one or more of these. Data analyzer 121 can be configured to identify self-limiting behavior of the subject and/or identify self-induced stress placed on the subject. Data analyzer 121 can be configured to determine the duration of exposure of the output data 131 to the subject (e.g. to determine if display of output data should be repeated). In some embodiments, data analyzer 121 is configured to determine the frequency of exposure of the output data to the subject. The data analyzer 121 can be configured to assess effectiveness of the behavior modification on the subject (e.g. an individual or group), such as by using artificial intelligence and/or computer learning to improve efficacy of the modification over time. In some embodiments, data analyzer 121 comprises one or more algorithms that are adjusted over time based on the assessed effectiveness. In some embodiments, one or more subjects are removed from a group of subjects based on the assessed effectiveness.

As described hereabove, data library 140 can store data recorded from input assembly 110 and/or it can include other data. For example, data library 140 can comprise data selected from the group consisting of: subject classifying data, such as age, race, religion, and/or sex of the subject; demographic data; data related to existing media that has been characterized to include a bias; empirical results of experience on altering a subject's bias (e.g. by system 10, such as to improve effectiveness of system 10); and combinations thereof.

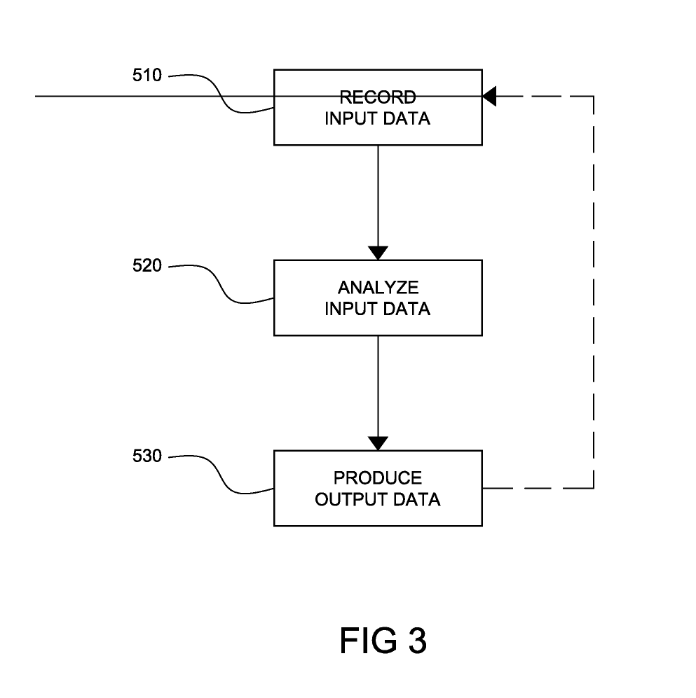

In some embodiments, data analyzer 121 is configured to produce output data 131 based on data analyzer 121's analysis of input data 111 and/or historical data 141, such as to modify the behavior of the subject. For example, data analyzer 121 may produce output data 131 as described herebelow in reference to FIG. 3.

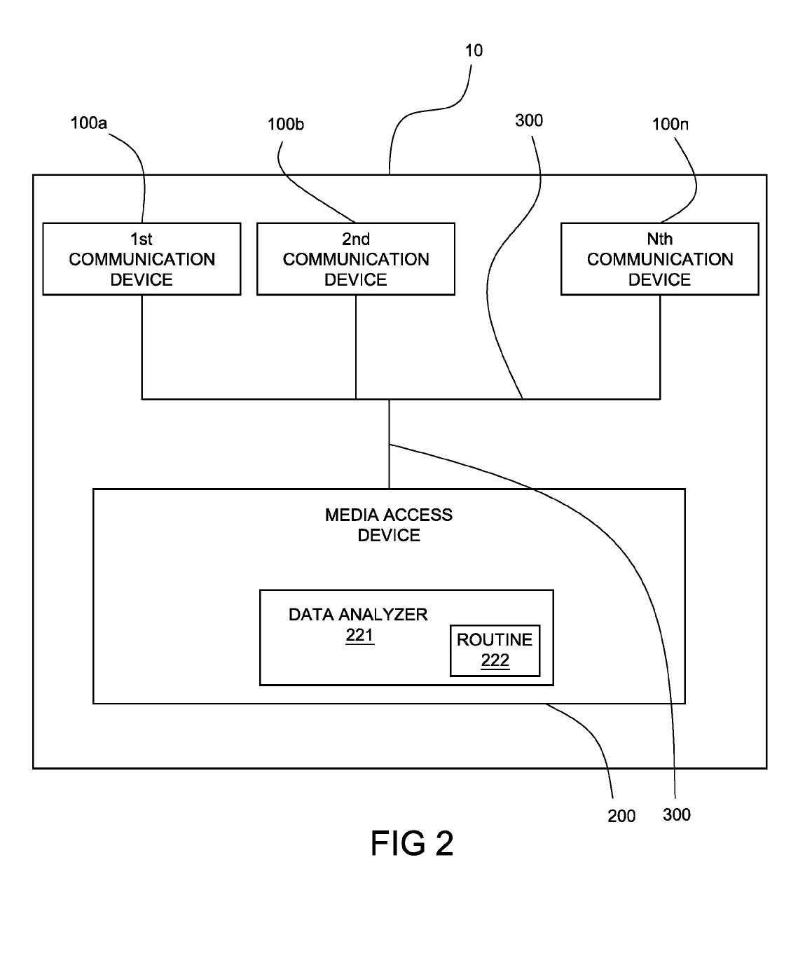

As described hereabove, system 10 can include one or more second communication devices, such as media access device 200 shown, which can transfer data to and/or from communication device 100 via data communication link 300. Data communication link 300 can comprise a network, such as a network selected from the group consisting of: the internet; wide area network; local area network; cellular service network; wired network; wireless network; and combinations thereof. Data communication link 300 can comprise a connection selected from the group consisting of: wired connection; wireless connection; Bluetooth connection; sneakernet; push and/or pull data transfer; and combinations thereof. In some embodiments, media access device 200 comprises one, two or more devices selected from the group consisting of: computer; network; the internet; cloud; disk drive; electronic file storage device; electronic library; and combinations of two or more of these. In some embodiments, media access device 200 comprises a device comprising living tissue that is configured to store information, such as information stored in the DNA of the tissue. The information can be accessed by sequencing the DNA. In some embodiments, media access device 200 is configured to analyze input data 111, such as in addition to or in the place of data analyzer 121, such as when media access device 200 comprises a data analyzer 221 as described herebelow in reference to FIG. 2. For example, media access device 200 can be at a remote location from communication device 100, connected to communication device 100 by data communication link 300 (e.g. a wired or wireless network), and media access device 200 can produce the output data 131 to be provided to the subject via output assembly 130. In some embodiments, media access device 200 comprises an input assembly, such as input assembly 110.

System 10 can be configured to provide output data 131 comprising a behavioral modification regimen for one or more subjects. For example, output data 131 can comprise a behavioral modification regimen configured to perform one, two or more functions selected from the group consisting of: modify prejudice of the subject; modify implicit bias of the subject; reduce, maintain (e.g. if at an acceptable level) and/or prevent creation of a detrimental bias of the subject; modify self-limiting behavior of the subject; identify harmful interactions of the subject with other people; identify harmful interactions of the subject with an environment; improve confidence of the subject; and combinations of one, two or more of these. Output data 131 can comprise a behavioral modification regimen that includes a report of bias measurements. Output data 131 can comprise a report of a “bias quotient” that includes an assessment of bias exposure of a subject. In some embodiments, output data 131 is stratified for one or more subjects that are included in a group of two or more subjects, such as to improve the efficacy of the behavioral modification provided by system 10.

Referring now to FIG. 2, a schematic view of a system for modifying behavior of a subject and including multiple communications devices attached to a media access device is illustrated, consistent with the present inventive concepts. System 10 comprises multiple communication devices, such as communication devices 100a, 100b through 100n shown (singly or collectively communication device 100). Each of the communication devices 100 can be operably connected to media access device 200 via data communication link 300. Each communication device 100, as well as media access device 200 and data communication link 300 can be of similar construction, arrangement and configuration as the similar components described hereabove in reference to FIG. 1. In the embodiment shown in FIG. 2, media access device 300 can comprise one or more computers or other data input and data output devices, and can include data analyzer 221 configured to analyze input data received from a communication device (e.g. using one or more algorithms of routine 222), and produce output data configured to modify behavior of a subject.

Referring now to FIG. 3, a flow chart of a method for producing output data configured to modify behavior of a subject based on an analysis of input data is illustrated, consistent with the present inventive concepts. The flow chart of FIG. 3 can be performed using system 10 and its various components, as described hereabove in reference to FIG. 1. In Step 510, input data is recorded, such as input data 111 recorded by an input assembly 110 of a communication device 100 as described hereabove.

In Step 520, the input data is analyzed, such as by data analyzer 121 of a communication device 100 as described hereabove in reference to FIG. 1, and/or via a data analyzer 221 of a media access device 200 as described hereabove in reference to FIG. 2.

In Step 530, output data is produced (e.g. by data analyzer 121 and/or data analyzer 221), such as output data provided (e.g. visually and/or audibly provided) to a subject comprising one or more subjects whose behavior is to be modified.

In some embodiments, system 10 is configured to repeat steps 510 through 530, such as when system 10 of the present inventive concepts is configured to modify behavior of a subject in a closed-loop fashion.

In some embodiments, one or more portions of one or more of steps 510 through 530 are modified, such as when data analyzer 121 and/or data analyzer 221 comprises artificial intelligence and/or computer learning configured to improve (e.g. continuously improve) the behavioral modification provided by system 10.

The foregoing description and accompanying drawings set forth a number of examples of representative embodiments at the present time. Various modifications, additions and alternative designs will become apparent to those skilled in the art in light of the foregoing teachings without departing from the spirit hereof, or exceeding the scope hereof, which is indicated by the following claims rather than by the foregoing description. All changes and variations that fall within the meaning and range of equivalency of the claims are to be embraced within their scope.

1. A system for modifying behavior of a subject, comprising:
an input assembly configured to receive input data;
a data analyzer configured to receive the input data from the input assembly, analyze the input data and produce a behavioral modification regimen based on the input data; and
an output assembly configured to receive the behavioral modification regimen from the data analyzer and produce output data based on the behavioral modification regimen;
wherein the output data is configured to modify behavior of the subject.

2.-78. (canceled)
